(19) United States
(12) Patent Application Publication
(10) Pub. No.: US 2019/0333607 A1
(43) Pub. Date: Oct. 31, 2019

(54) DISEASE-ORIENTED GENOMIC ANONYMIZATION

(71) Applicant: KONINKLIJKE PHILIPS N.V., EINDHOVEN (NL)

(72) Inventors: Daniel Pletea, Eindhoven (NL); Tim Hulsen, Den Bosch (NL); Wilhelmus Petrus Maria van der Linden, Schijndel (NL); Peter van Liesdonk, Eindhoven (NL)

(21) Appl. No.: 16/310,065

(22) PCT Filed: Jun. 19, 2017
(86) PCT No.: PCT/EP2017/064863

§ 371 Date: Dec. 14, 2018

(30) Foreign Application Priority Data
Jun. 29, 2016 (EP) 16176810.6

(51) Int. Cl. G16B 50/40; G16B 20/00; G16B 40/00; G06F 21/62

(52) U.S. Cl. CPC G16B 50/40; G06F 21/6254; G16B 40/00; G16B 20/00

(57) ABSTRACT

A method, a system and a computer program product for anonymization of genetic data from at least one individual wherein the genetic data are grouped into a subset of genetic data being directly related to a disease and one or more subsets of genetic data being distantly related to the disease based upon the genome pathways network, and wherein the subsets of genetic data being distantly related to the disease are anonymized.

### FIELD OF THE INVENTION

The present invention relates to the analysis of genetic data. More specifically, the present invention relates to the analysis of genetic data with respect to a specific disease or disorder.

### BACKGROUND OF THE INVENTION

Nowadays medical and health records of patients are collected and used for clinical bioinformatics research. Next to clinical data, imaging data or biobanking data of patients also their genetic data are collected, and analyzing genetic data plays a significant role in medical research and in diagnostics and anamnesis. For example, the patients' genetic data are analyzed for finding or improving treatments for different diseases.

However, analysis of their genetic data might pose threats for the patients that are sharing their genetic data in that, for examples, their privacy will be violated. The violation is due to the fact that the genome of a person contains data such as those concerning eye colour, skin colour. These genetic data, together with other data embedded in the genome of a person, can lead to identification of a person by analyzing their genetic data. In order to protect the privacy of individuals, certain parts of the person's genome need to be anonymized when the genetic data are provided for medical bioinformatics research and analysis.

Some of the existing solutions for genome anonymization in bioinformatics research attempt to anonymize the entire genome, without taking in consideration the disease to be studied. As anonymization means loss of information, these existing solutions lead also to loss of information with respect to the genes which are directly related to the disease to be studied, which is not desirable.

Other solutions for genome anonymization consider a forensics context, which is a different type of attack model than the one addressed by this invention.

Furthermore given that genetic analysis is more widely adopted, a patient's consent is limiting the collection of his/her genomic information only to a subset of genes without a flexible anonymization solution. This subset of genes might later, during research, turn out as being too limited and that a related gene would be useful for analysis. Even though the person might have given consent to the use of this related gene as it is still disease related, the gene is already missing from the dataset due to earlier privacy concerns.

Moreover, while hiding some of the patient's genetic information, an anonymization techniques should also enable discovering when the set of disease-related genes needs to be modified, especially when the set of disease-related genes needs to be enlarged.

US 2014/0236833 A1 discloses a method for establishing a transaction between an individual and a third party, based on the genetic identity of an individual, wherein the individual allows the third party to access and analyze only a subset of the genetic identity required for the offer and establishment of the transaction.

US 2010/0063843 A1 discloses a computer based method and system for masked data record access in which data masks are applied to sensitive personal information so that non-masked portions of that information can be used in the selection of products, services and service providers for a consumer.

### SUMMARY OF THE INVENTION

In order to address the problems described above a solution is proposed, where the genetic data of the genome of one or more individuals are separated into different layers, based on how closely related the genetic data are with the genes relevant to the disease to be studied. This relationship is established based on the genome's pathways network. Different anonymization techniques are then used for anonymizing the layers of genetic data other than the genetic data being directly related to the disease to be studied. The anonymization techniques that are used are chosen for each layer of genetic data, based on its estimated relevance. The genetic data directly related to the disease to be studied remain unanonymized and can be used for analysis.

### BRIEF DESCRIPTION OF THE DRAWINGS

In the drawings:

FIG. 1 represents a schematic illustration of layering genetic data for disease-oriented anonymization.

FIG. 2 represents a schematic illustration of re-layering genetic data.

FIG. 3 is a flow chart illustrating the steps of an embodiment of the method of the layered disease-oriented anonymization.

FIG. 4 illustrates an example of a computer readable medium for storing a computer executable code for implementing the method for anonymizing genetic data.

FIG. 5 illustrates an embodiment of a system which is configured for anonymizing genetic data.

### DETAILED DESCRIPTION OF EMBODIMENTS

In a first aspect, the invention provides a method for anonymization of genetic data.

In a second aspect, the invention provides a computer program product providing anonymization of genetic data.

In a third aspect, the invention provides a system for anonymization of genetic data.

In a fourth aspect, the invention provides the use of the method and/or the computer program product for bioinformatics research and/or for diagnostics.

The present invention will be described with respect to particular embodiments and with reference to the figures, but the invention is not limited thereto, but only to the claims. The drawings described are only schematic and are non-limiting. In the drawings, the size of some of the elements may be exaggerated and not drawn on scale for illustrative purposes.

According to the first aspect, the present invention provides a method for anonymization of genetic data from at least one individual with respect to a specific disease. Said method for anonymization of genetic data comprises the steps of:

providing genetic data from at least one individual;

choosing a disease to be studied;

determining subset(s) of genetic data from the genetic data of the at least one individual being directly related to the disease to be studied;

assorting the subsets of the genetic data that are not directly related to the disease to be studied into different layers based on the subsets' distance to the genetic data being directly related to the disease to be studied; and

anonymizing the layers that are not directly related to the disease to be studied or the genetic data present in the layers that are not directly related to the disease to be studied.

In the method, genetic data from at least one individual are used. The term “genetic data” refers to any kind of genetic information. The term “genetic data” includes the nucleotide sequence of the individuals' genome or of a portion of the individuals' genome. “Genetic data” also includes genetic information other than a nucleotide sequences as such, for example information on the presence or absence of genetic markers such as, for example Amplified Fragment Length Polymorphisms (AFLPs), Randomly Amplified Polymorphic DNA (RAPD), Restriction Fragment Length Polymorphisms (RFLPs), Single Nucleotide Polymorphisms (SNPs), Short Tandem Repeats (STRs) and Variable Number Tandem Repeats (VNTRs). The term “genetic data” also comprises information concerning RNA and proteins. Thus, the term “genetic data” comprises information concerning nucleotide sequences, amino acid sequences, structure, activity, abundance and/or function of nucleic acid molecules and/or proteins. In addition, “genetic data” comprises copy number data, such as data on copy numbers of genes or other nucleotide sequence stretches.

The term “individual” refers to a human subject. Said human subject may or may not be affected by/suffering from the disease to be studied. Hence, the terms “individual”, “person” and “patient” are synonymously used in the instant disclosure.

The expression “providing genetic data” is understood that the genetic data of at least one individual need to be obtained. However, the genetic data of the at least one individual do not have to be obtained in direct association with the method or for performing the method. Typically the genetic data of the at least one individual are obtained at a previous point or period of time, and are stored electronically in a suitable electronic storage device and/or database. For performing the method, the genetic data can be retrieved from the storage device or database and utilized.

The expression “choosing a disease to be studied” denotes that the method can be used to study or analyze any disease, disorder or medical condition. Hence, a particular disease, disorder or medical condition has to be chosen or defined for subsequently determining the subset of genetic data being directly related to said disease, disorder or medical condition, and the genetic data not directly related to said disease, disorder or medical condition.

The term “directly related” with respect to the relation of the subset of genetic data and the disease to be studied, refers to genetic loci and/or genes which cause said disease or are in straight line with said genetic loci and/or genes causing the disease. The genetic loci and/or genes comprise protein coding regions (open reading frames) as well as non-protein coding regions upstream or downstream of an open reading frame. Said genetic loci and/or genes also comprise those that are directly involved in regulating the expression of the genes that cause the disease to be studied. Hence, “directly related” includes structural features of the protein coding regions of those genes encoding proteins or polypeptides causing the disease to be studied as well as those elements directly involved in regulating the expression of the genes encoding proteins or polypeptides causing the disease.

The term “layer” refers to a sub-group of genetic data that are not directly related to the disease to be studied. A layer may comprise a plurality of subsets of genetic data. For example, a layer is a subset of genes which have the same distance to any of the directly disease related core genes, wherein two different layers have two different such distances. Each layer is assigned an anonymization method, wherein multiple layers can be assigned the same anonymization method.

In an embodiment, the method for anonymization of genetic data is intended for studying a particular disease by bioinformatics means, i.e. by using software tools for an in silico analysis of biological queries using mathematical and statistical techniques to analyze and interpret biological data with respect to their relevance for the particular disease. This embodiment typically requires use of genetic information of a plurality of individuals.

In another embodiment of the method for anonymization of genetic data, the method is intended for use in diagnostics, wherein the genetic information of an individual is analyzed for the genetic disposition and/or occurrence of a specific disease or disorder of said individual.

The method can be applied to any disease, disorder or medical condition. The disease to be studied is a specific disease that is chosen on purpose. In an embodiment, the disease to be studied is known to be a disease that is associated with a particular genotype. Examples of such diseases are cancers, immune system diseases, nervous system diseases, cardiovascular diseases, respiratory diseases, endocrine and metabolic diseases, digestive diseases, urinary system diseases, reproductive system diseases, musculoskeletal diseases, skin diseases, congenital disorders of metabolism, and other congenital disorders such as prostate cancer, diabetes, metabolic disorders, or psychiatric disorders.

In the method, the genetic data of said at least one individual are grouped into subsets or layers of genetic information based on the relation of the genetic data to the disease to be studied. Thus, those genetic data known to be directly related to the disease to be studied (the core-disease genes) are grouped into a subset which is not anonymized.

“Genetic data” directly related to the disease to be studied comprise the gene(s), markers, RNA and proteins that are connected to the disease to be studied, preferably in that the sequence, structure, activity, abundance and/of function of the subject matter of said genetic data either causes the disease to be studied or is a direct consequence of the disease to be studied. The genetic data might concern the nucleotide sequence of one or more genes, either within the protein coding region and/or outside the protein coding region. The genetic data may concern regulatory genes as well. The genetic data directly related to the disease to be studied are put into a sub-group that may be designated “the core”.

Genetic data that are not directly related to the disease to be studied are grouped into at least one subset or layer. Theoretically, the number of layers may be as high as x−1, wherein x represents the number of genes in a given genome. Preferably the genetic data that are not directly related to the disease to the studied are grouped into one of two or more layers, based on the degree of their distance from one or more of the core-disease genes, wherein the closest distance is selected if the subset of genetic data has different distances to different core-disease genes. In an embodiment, the number of subsets or layers is equal or less than 10, preferably the number of subsets/layers is 2, 3, 4, 5, 6, 7, 8, 9, or 10. Hence, in an exemplary embodiment wherein the number of layers is 1, the genetic data are split into directly disease-related data and not directly disease-related data or not disease-related data. In alternative embodiments, wherein the number of layers is 2 or more, the genetic data are split into a directly disease-related data subset and several subsets of not directly disease-related data.

For determining the relation of a subset of genetic data to the disease to be studied and/or its relative distance to the subset of genetic data directly related to the disease to be studied, the genome pathway networks are utilized.

Genome pathway networks are available and accessible via databases on the internet, and may be established—for example—for a specific disease such as prostate cancer (http://www.genome.jp/dbget-bin/www_bget?pathway:map05215), type II diabetes mellitus (http://www.genome.jp/dbget-bin/www_bget?pathway:map04930) or Parkinson's disease (http://www.genome.jp/dbget-bin/www_bget?pathway:map05012).

In an additional and/or alternative embodiment, the genome pathway networks are not established with respect to a specific disorder. Examples for such more generic genome pathway networks databases are the Reactome open-source curated and peer reviewed pathway database (www.reactome.org), the BioCyc Database Collection of Pathway//Genome Databases (www.biocyc.org), the Pathway Commons pathway information database (www.pathwaycommons.org), and the databases of the Gene Ontology Consortium (www.geneontology.org).

In an additional and/or alternative embodiment, the STRING database (https://www.string-db.org) is utilized. STRING is a database of known and predicted protein-protein interactions. The interactions include direct (physical) and indirect (functional) associations; they stem from computational prediction, from knowledge transfer between organisms, and from interactions aggregated from other (primary) databases. Interactions in the STRING database are derived from genomic context predictions, high throughput lab experiments, (conserved) coexpression of genes, automated text mining and previous knowledge in databases. The STRING database covers 9′643′763 proteins from 2′031 organisms at the end of June 2016. The STRING database is operated by the STRING Consortium which includes the Swiss Institute of Bioinformatics, the CPR-NNF Center for Protein Research, and the European Molecular Biology Laboratory.

The genetic data directly related to the disease to be studied and present in the core layer are not anonymized and thereby available for analysis without restrictions.

The genetic data and/or the layers of the genetic data not directly related to the disease to be studied are anonymized by using techniques that are selected from the group consisting of statistical anonymization, encryption, and secure multiparty anonymization and computation.

These anonymization techniques allow analysis on the data, but this analysis is limited due to their properties. The statistical anonymization implies loss of information, but keeps the rest of the information in a human-readable shape. This allows analyses to be performed on the data, but the results are limited by the loss of information from the beginning Encryption techniques do not lose information, but this information is not available. However, if there is ever any indication that the encryption information is necessary for research, a privacy officer is able to extend the core disease information by decrypting this set. An intermediate solution exists where modern techniques like homomorphic encryption, multi-party computations and/or other operations on encrypted data are used to combine the core disease set with the encrypted layers. In these situations the privacy-sensitive information will stay secret, while the result of these operations can be disclosed by the privacy officer. These techniques insert latency in the analysis and therefore are limiting the possible analyses that can be performed on the data.

In an embodiment, the statistical anonymization is selected from the group consisting of k-anonymity, l-diversity, t-closeness and δ-presence.

K-anonymity is a formal model of privacy created by L. Sweeney. The goal is to make each record indistinguishable from a defined number (k) of other records if attempts are made to identify the data. A set of data is k-anonymized if, for any data record with a given set of attributes, there are at least k−1 other records that match those attributes [J. Sedayao, “Enhancing Cloud Security Using Data Anonymization,” June 2012. [Online]. Available: http://www.intel.nl/content/dam/www/public/us/en/documents/best-practices/enhancing-cloud-security-using-data-anonymization.pdf. (Accessed 26 Jan. 2015)], [L. Sweeney, “K-anonymity: A Model for Protecting Privacy,” Int. J. Uncertain. Fuzziness Knowl.-Based Syst., vol. 10, no. 5, pp. 557-570, 2002.]. A typical value fork is 3 [M. Templ, B. Meindl, A. Kowarik and S. Chen, “Introduction to Statistical Disclosure Control (SDC),” August 2014. [Online]. Available: http://www.ihsn.org/HOME/sites/default/files/resources/ihsn-working-paper-007-Oct27.pdf. (Accessed 26 Jan. 2015)]. L-diversity improves anonymization beyond what k-anonymity provides. The difference between the two is that while k-anonymity requires each combination of quasi identifiers to have k entries, l-diversity requires that there are 1 different sensitive values for each combination of quasi identifiers [J. Sedayao, “Enhancing Cloud Security Using Data Anonymization,” June 2012. [Online]. Available: http://www.intel.nl/content/dam/www/public/us/en/documents/best-practices/enhancing-cloud-security-using-data-anonymization.pdf. [Accessed 26 Jan. 2015]] [4].

T-closeness requires that the distribution of a sensitive attribute in any equivalence class is close to the distribution of the attribute in the overall table (i.e., the distance between the two distributions should be no more than a threshold T) [N. Li, T. Li and S. Venkatasubramanian, “t-Closeness: Privacy Beyond k-Anonymity and I-Diversity,” in Data Engineering, 2007. ICDE 2007. IEEE 23rd International Conference on, 2007.]. L-diversity requirement ensures “diversity” of sensitive values in each group, but it does not take into account the semantically closeness of these values. This is done by t-closeness.

δ-presence is a metric to evaluate the risk of identifying an individual in a table based on generalization of publicly known data. δ-presence is a good metric for datasets where “knowing an individual is in the database poses” a privacy risk. [M. E. Nergiz, M. Atzori and C. Clifton, “Hiding the Presence of Individuals from Shared Databases,” in Proceedings of the 2007 ACM SIGMOD International Conference on Management of Data, Beijing, China, 2007.]

The anonymization techniques “searchable encryption”, “homomorphic encryption”, and “secure multiparty computation” have the advantage that decryption of the encrypted data is not actually necessary, but it is feasible to perform data processing in the encrypted domain. The main difference between these techniques is the choice of trade-offs they make. Searchable encryption limits the processing to a simple keyword match. Fully homomorphic encryption can do any kind of processing, but has extremely big ciphertext sizes and is computationally very intensive. Multiparty computation scales better, but requires non-colluding computers to work together to do the processing.

In an additional and/or alternative embodiment, the genetic data and/or the layers of the genetic data not directly related to the disease to be studied are anonymized by encryption, preferably selected from the group consisting of homomorphic encryption, searchable encryption and non-malleable encryption.

In comparing with gene removal, the non-malleable encryption has the advantage that the data is not lost and the statisticians can notice the presence of more data in certain direction of the genome. Furthermore, when noticed that a certain gene should have been categorized as a core-disease gene, a new layering of the genome can be created and the genome re-anonymized according to the new set of core-disease genes.

In an additional and/or alternative embodiment, the anonymization considers proximity of the genetic data within a layer to the core in that layers containing genetic data which are closer to the core disease are anonymized using techniques which involve losing less information and thus still allow some degree of analysis.

In an additional and/or alternative embodiment, the different layers are anonymized by different techniques, preferably depending on the distance of the layers' subsets of genetic data to the subset of genetic data being directly related to the disease to be studied. Anonymizing the different layers by different techniques improves data security as it becomes more difficult to inadvertently decode the genetic data.

The properties of genetic information anonymized by the method disclosed herein are detectable, since at least one subset—the core layer—is readable by humans. The subsets of genetic data being statistically anonymized data are readable by humans. In addition, the statistically anonymized data can be detected by using tools which are verifying if the data has properties like 2-anonymity. In an embodiment, said tool is selected from the group consisting of ARX-Anonymization Tool, UTD Anonymization Toolbox, μ-Argus, R-Package sdcMicro, Cornell Anonymization Toolkit, PARAT, CATS de-identification platform, IRI FieldShield, Gedis Studio Anonymization, SAFELINK, ANU Data Mining Group, Data Swapping Toolkit, Ruby data anonymization tool and Reversible log anonymization tool.

The ARX Data Anonymization Tool (http://arx.deidentifier.org/anonymization-tool/) can be used to check whether the data is correctly anonymized by comparing the output with the input, which should not differ if the data is in CSV format. The UTD Anonymization Toolbox (http://cs.utdallas.edu/dspl/cgi-bin/toolbox/index.php) covers the anonymization models: k-anonymity, l-diversity, t-closeness. It can be used in the same manner as ARX Data Anonymization Tool.

The μ-Argus (Anti-Re-Identification General Utility System) is a software package which was developed at Statistics Netherlands (http://neon.vb.cbs.nl/casc/Software/MuManual4.2.pdf). This software package is providing risk-approach, post randomization (PRAM), numerical micro-aggregation, ranks swapping. The code is available here: http://neon.vb.cbs.nl/casc/mu.htm.

The R-Package sdcMicro is an R package tool. It can be used for generation of anonymized microdata. The tool can be downloaded from: http://cran.r-project.org/web/packages/sdcMicro/. sdcMicro contains almost all popular methods for the anonymization of both categorical and continuous variables. This tool is using GPL license.

The Cornell Anonymization Toolkit (CAT) (http://sourceforge.net/projects/anony-toolkit/) implements two privacy criteria: l-diversity and t-closeness. Given a certain privacy criterion, there are a number of anonymization strategies to achieve this criterion, such as data generalization, data swapping, data perturbation, etc. CAT currently supports only the data generalization mechanism.

PARAT (http://www.privacyanalytics.ca/software/) is an integrated de-identification and masking software focused on health data. It is commercially available. PARAT can handle structured data and unstructured data and is using different protection methods: masking, de-identification for different types of variables: direct identifiers, quasi identifiers.

The CATS de-identification platform (https://www.custodix.com/index.php/cats) CATS (Custodix Anonymisation Services) is a service-oriented platform for de-identification of data. CATS support anonymization of different types of data (CSV, XML, HL7, DICOM) in a generic and extendable way. It can be integrated into automated data-flows or be used for manual de-identification.

The IRI FieldShield (http://www.iri.com/solutions/data-masking/de-identification/anonymize) provides functions for de-identification, encoding, encrypting, data masking, randomization and pseudonymization.

The Gedis Studio Anonymization (http://www.gedis-studio.com/anonymization.html) provides anonymization with data encryption and scrambling, but also with data masking. The data masking can be done while taking in consideration the data distribution.

SAFELINK (https://www.uni-due.de/soziologie/schnell_forschung_safelink_software.php) is a specification and implementation of a privacy-preserving record-linkage procedure, which uses cryptographic hashing (keyed HMACs).

The ANU Data Mining Group (http://datamining.anu.edu.au/projects/linkage.html) is aiming at developing techniques for blindfolded record linkage, based one-way hashing and/or encryption.

The Data Swapping Toolkit can be found here (http://www.niss.org/sites/default/files/dstk-afk.pdf)

The Ruby data anonymization tool (https://www.ruby-toolbox.com/projects/data-anonymization) is using the whitelist and blacklist concepts for dealing with removal of direct identifiers. The code can be found here: https://github.com/sunitparekh/data-anonymization.

The Reversible log anonymization tool (http://blog.cassidiancyber-security.com/post/2014/01/Reversible-log-anonymization-tool) is a tool designed to replace sensitive fields in customer's logs with anonymized values, while generating a lookup table. In an additional and/or alternative embodiment, the subsets of encrypted data allow comparison on the cipher-text and therefore revealing information which can be used in the analysis of the disease to be studied. The analysis of the encrypted data can be detected
via database data-retrieval analysis, wherein the encrypted data from the database is selected and used locally in other parts of the system which are performing operations on the encrypted data; and/or
via traffic analysis, which reveals multi-party computations performed on other machines than the local one.

The method is advantageous due to its flexible anonymization. The method allows de-anonymization and re-anonymization of the genetic data. Based on the progress in research, previously anonymized genetic data can be recovered and newly assorted, either by the same process and entity that performed the first anonymization, or by a third party.

In an alternative and/or additional embodiment, the method further comprises analyzing the genetic data directly related to the disease to be studied. Typically, the analysis of the genetic data with respect to the disease to be studies has to be performed by another entity than the one anonymizing the genetic data.

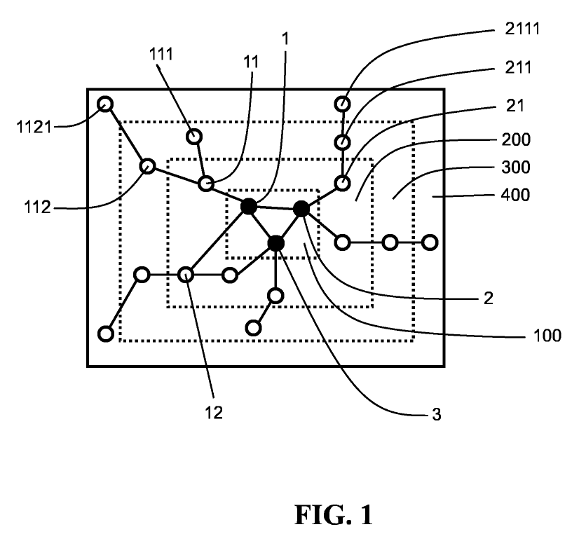

Referring to FIG. 1, a layered disease-oriented anonymization of genetic data is illustrated. In this embodiment, the genetic data are deemed to be genes. Each gene is represented by a circle. The genes directly related to the disease to be studied are the core genes (1, 2, 3) and are present in the core (100). These core genes are shown as solid circles. Three layers (200, 300, 400) are provided for bearing genes that are not directly related to the disease to be studied. The genes that are not directly related to the disease to be studied are shown as open circles. Genes 11 and 12 are in straight line to core gene 1 as illustrated by the solid lines between the circles representing the respective genes. Genes 11 and 12 are grouped in layer 1 (200) which bears those genes that are in closest proximity to the core genes, but which are not directly related to the disease to be studied. Genes 111 and 112 are in straight line to gene 11, but are less closely related to core gene 1. Therefore, genes 111 and 112 are put into layer 2, containing genes that are more distantly related to the core genes than the genes in straight line to the core genes. The layers 200, 300, 400 and the genes contained in said layers are anonymized, wherein the core 100 and the core-disease gene 1, 2, 3 are not anonymized.

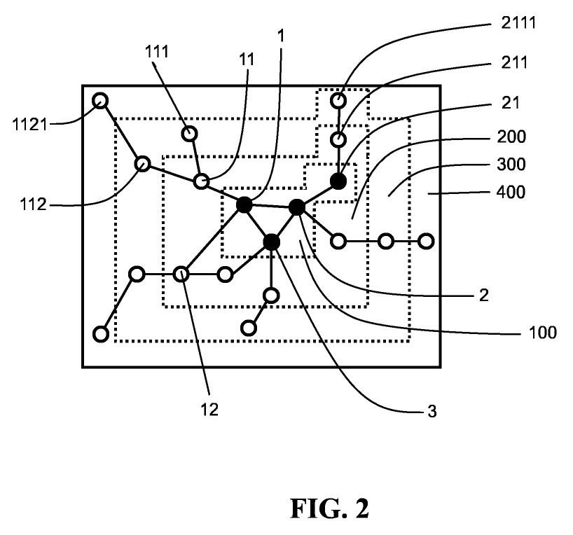

FIG. 2 illustrates the layered disease-oriented anonymization as shown in FIG. 1 after de-anonymization and re-anonymization for including gene 21 as core gene being directly related to the disease to be studied. As shown in FIG. 1, gene 21 was initially considered a gene in straight line to core gene 2, but not being directly related to the disease to be studied. If gene 21 will be understood to be directly related to the disease to be studied due to progress in research and development, it is included into the core 1 as shown in FIG. 2. In addition, gene 211 being in straight line to gene 21, will also be moved into the layer next closer to the core, namely moving from layer 300 to layer 200, wherein the layers 200, 300, 400 and the genes contained in said layers are anonymized, but the core 100 and the core-disease gene 1, 2, 3, 21 are not anonymized. Hence, any gene being in straight line to a given gene, i.e. where the gene or the polypeptide encoded by said gene directly interacts with another gene or the polypeptide encoded by said another gene, is assorted to the layer being one layer closer to the core if said given gene is determined to be a core disease gene. Assorting of said another genes being in straight line with said given gene into the layer being one layer closer to the core occurs due to the direct interaction of genes and/or polypeptides encoded by said genes.

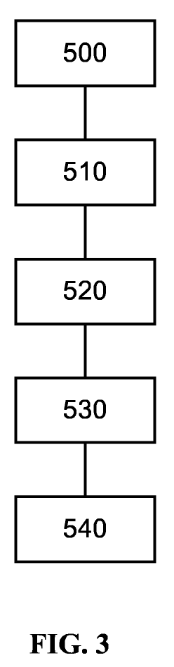

FIG. 3 represents a schematic flow chart illustrating an embodiment of the method for disease oriented anonymization of genetic data, wherein step 500 represents collecting and storing genetic data of one or more individuals. In step 510 the disease to be studied is chosen. Then the core-disease genes are determined in step 520 and the genes are assorted into different layers based on the genome pathways network and the proximity of the genes to the core-disease genes. In step 540, the genetic data present in the layers other than the core layer are anonymized.

According to the second aspect, the invention provides a computer program product for anonymizing genetic data. The computer program product comprises instructions which when carried out on a computer cause the computer to perform at least one step of a method for anonymizing genetic data of at least one individual, the method comprising the steps of:

providing genetic data from at least one individual;

choosing a disease to the studied;

determining at least one subset of the genetic data, said subset of genetic data being directly related to the disease to be studied;

assorting the remaining genetic data which are not directly related to the disease to be studied into multiple subsets grouped into more than one layer based on the proximity of these subsets to the genetic data which are directly related to the disease to be studied, wherein the proximity is preferably established based on a genome pathway network that corresponds to the genetic data;

anonymizing the more than one layer containing the subsets of genetic data not directly related to the disease to be studied.

In an embodiment, the computer program product comprises instructions which when carried anonymizes the one or more layers containing the subsets of genetic data not directly related to the disease to be studied. Anonymization of the one or more layers is performed by using at least one technique selected from the group consisting of statistical anonymization, encryption and secure multiparty anonymization and computation as described herein before with respect to the first aspect of the invention.

In an additional and/or alternative embodiment, the computer program product comprises instructions which when carried out assorts the remaining genetic data which are not directly related to the disease to be studied into one or more subsets and into one or more layers based on the proximity of these subsets to the genetic data which are directly related to the disease to be studied.

In an additional and/or alternative embodiment, the computer program product comprises instructions which when carried out determines at least one subset of the genetic data, said subset of genetic data being directly related to the disease to be studied.

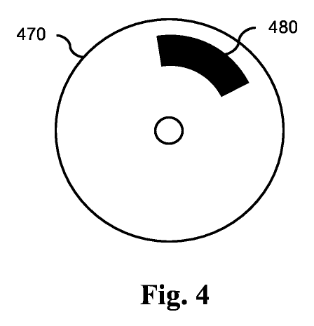

In an embodiment, the method as described in FIG. 3 may be implemented on a computer as a computer implemented method, as dedicated hardware, or as a combination of both. As also illustrated in FIG. 4, instructions for the computer, e.g., executable code, may be stored on a computer readable medium 470, e.g., in the form of a series 480 of machine readable physical marks and/or as a series of elements having different electrical, e.g., magnetic, or optical properties or values. The executable code may be stored in a transitory or non-transitory manner. Examples of computer readable mediums include memory devices, optical storage devices, integrated circuits, servers, online software, etc. FIG. 4 shows an optical disc 470.

It will be appreciated that the invention applies to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

According to the third aspect, the invention provides a system for anonymizing genetic data. Said system comprises

a data interface configured to receive genetic data of at least one individual;

a user input interface configured to receive user input commands form a user input device for choosing a disease to be studied; and

a processor configured for:

determining subset(s) of genetic data from the genetic data of the at least one individual being directly related to the disease to be studied;

assorting the subsets of the genetic data that are not directly related to the disease to be studied into different layers based on the subsets' distance to the genetic data being directly related to the disease to be studied, wherein the distance is preferably established based on a genome pathway network that corresponds to the genetic data; and

anonymizing the layers that are not directly related to the disease to be studied or the genetic data present in the layers that are not directly related to the disease to be studied.

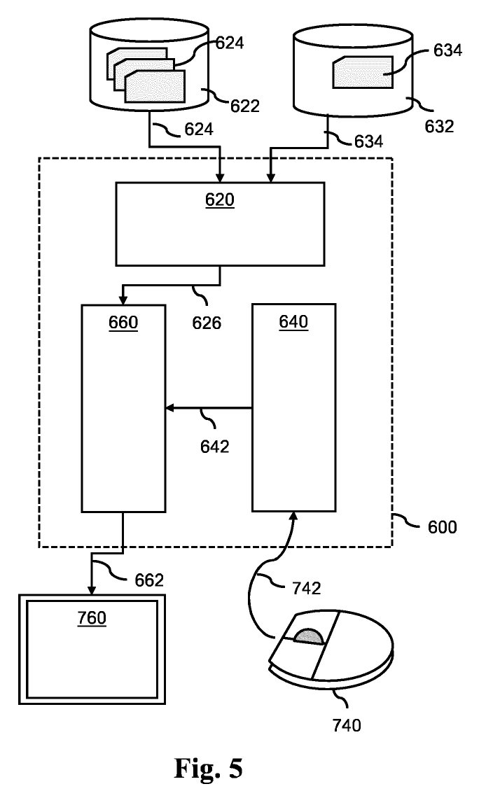

FIG. 5 shows a system 600 which is configured to anonymizing genetic data. The system 600 comprises a data interface 620 configured to access genetic data 624 of at least one individual. The data interface 620 is further in communicative with database 634 of a Genome pathway networks 632. In the example of FIG. 6, the data interface 620 is shown to be connected to an external repository 622, such as a suitable electronic storage device and/or database, which comprises the genetic data 624 of the at least one individual. The data interface 620 is further connected to a Genome pathway network 632. Alternatively, the genetic data 624 of the at least one individual as well as the database 634 may be accessed from an internal data storage of the system 600. In general, the data interface 620 may take various forms, such as a network interface to a local or wide area network, e.g., the Internet, a storage interface to an internal or external data storage, etc.

Furthermore, the system 600 is shown to comprise a user input interface 640 configured to receive user input commands 742 from a user input device 740 to enable the user to provide user input, such as choose or define a particular disease, disorder or medical condition for subsequently determining the subset of genetic data being directly related to said disease, disorder or medical condition, and the genetic data not directly related to said disease, disorder or medical condition, choose or select the genome pathway networks 632 that correspond to the selected genetic data. The user input device 740 may take various forms, including but not limited to a computer mouse, touch screen, keyboard, etc. FIG. 5 shows the user input device to be a computer mouse 740. In general, the user input interface 640 may be of a type which corresponds to the type of user input device 740, i.e., it may be a thereto corresponding user device interface.

The system 600 is further shown to comprise a processor 660 configured to determine at least one subset 100 of the genetic data 624, said subset 100 of genetic data 624 being directly related to the disease to be studied; assort the remaining genetic data which are not directly related to the disease to be studied into one or more subsets and into one or more layers (200, 300, 400) based on the proximity of these subsets to the genetic data which are directly related to the disease to be studied; and anonymize the one or more layers containing the subsets of genetic data not directly related to the disease to be studied.

The processor 660 is configured to determine the relation of a subset of genetic data to the disease to be studied and/or its relative distance to the subset of genetic data directly related to the disease to be studied by utilizing the genome pathway networks 632.

Genome pathway networks 632 are available and accessible via databases on the internet, and may be established—for example—for specific disease such as prostate cancer type II diabetes mellitus or Parkinson's disease.

In an example, based on the received user input commands 742, the processor 660 may transmit the genetic data 624 of the at least one individual to the selected genome pathway networks 632 via the data interface 620. In return, the processor 660 may receive a result indicating the relation of a subset of genetic data to the disease to be studied and/or its relative distance to the subset of genetic data directly related to the disease to be studied from the genome pathway networks 632. Subsequently, the processor 660 may further group the genetic data of said at least one individual into subsets or layers of genetic information based on received result indicating the relation of the genetic data to the disease to be studied. Thus, those genetic data known to be directly related to the disease to be studied (the core-disease genes) are grouped by the processor 660 into a subset 100. The genetic data and/or the layers (200, 300, 400) of the genetic data not directly related to the disease to be studied are grouped subsequently based on its relative distance to the subset of genetic data directly related to the disease to be studied. Here, the ‘distance’ between two genes is determined by some types of interaction. Such interaction can be coexpression, protein-protein interaction, copublication, etc., or any combination thereof. For instance, the STRING database lists a few possibilities of interaction (http://www.string-db.org/help/getting_started/#evidence).

The processor 600 is further configured to anonymize the genetic data and/or the layers (200, 300, 400) of the genetic data not directly related to the disease to be studied by selecting one or more algorithms from a group of algorithms consisting of statistical anonymization, encryption, and secure multiparty anonymization and computation. The group of algorithms is stored in a memory 670 (not shown in FIG. 5).

In a preferred example, the database 634 may be included in the system 600. Accordingly, based on the received user input commands 742, the processor 660 may receive the genetic data 624 of the at least one individual from the external repository 622. The processor 660 may further determine subset(s) of genetic data in association with the database 634. Subsequently, the processor may assort the subsets of the genetic data that are not directly related to the disease to be studied into different layers based on the subsets' distance to the genetic data being directly related to the disease to be studied. Later, the processor 660 may anonymize the layers that are not directly related to the disease to be studied or the genetic data present in the layers that are not directly related to the disease to be studied. A detailed example showing how the subsets of the genetic data are assorted and anonymized can be found below.

The processor 600 is further configured to generate anonymized genetic data 662 to an output device 760, such as a display. Alternatively, the display 760 may be an internal part of the system 600.

Alternatively, the processor 600 may be configured to automatically choose or define a particular disease, disorder or medical condition for subsequently determining the subset of genetic data being directly related to said disease, disorder or medical condition, and the genetic data not directly related to said disease, disorder or medical condition, as well as automatically choose or select the genome pathway networks 632 that correspond to the selected genetic data.

According to the fourth aspect, the invention concerns the use of the method and/or the computer program product in bioinformatics research and/or in diagnosis.

In an embodiment, the method and/or computer program product is used in bioinformatics research. The use of the method and/or computer program product in bioinformatics research comprises acquiring the genetic data of a plurality of individuals. Examples of research fields in bioinformatics the use of the method and/or the computer program product in bioinformatics research can be applied to and which are encompassed by the fourth aspect are genomics, genetics, transcriptomics, proteomics and systems biology.

In an alternative embodiment, the method and/or computer program product is used in diagnosis, wherein the genetic data of an individual are utilized to analyze whether the individual is affected by a specific disease or at risk of getting said disease or being affected by said disease.

The present invention can be applied in the diagnostics domain and the genomics domain, wherein the genetic data of the individuals are organized in a hierarchy with a core set of data that are immediately available for further analysis, and layers of increasing sensitivity that can either be revealed or used in computation with encrypted data. The present invention improves the individuals' consent gathering process for the individuals as well as for the owner of the data. The individuals are sure that their genetic data are properly anonymized, while allowing re-anonymization triggered by progress in research. Thereby, it becomes easier to define the individuals' consent, by allowing access to “the genetic data relevant for performing research on the disease to be analyzed or studied”.

Where an indefinite or definite article is used when referring to a singular noun, e.g. “a”, “an”, “the”, this includes a plural of that noun unless something else is specifically stated. Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Moreover, the terms top, bottom, over, under, beyond and the like in the description and in the claims are used for descriptive purposes and not necessarily for describing relative positions. It is to be understood that the terms so used are interchangeable under appropriate circumstances and that the embodiments of the invention described herein are capable of operation in other orientations than described or illustrated herein. It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

### EXAMPLE

Disease Oriented Genomic Anonymization with Respect to Prostate Cancer

In a first step, a list of the core prostate cancer genes were retrieved by looking into the KEGG pathway database (http://www.genome.jp/dbget-bin/www_bget?pathway:map05215) for the prostate cancer pathway.

A total of 70 genes that are part of this pathway were retrieved using the KEGG Orthology because this database groups all genes belonging to multiple species into orthologous groups, removing any redundancy. These 70 genes are all genes that are deemed to be directly related to prostate cancer. These 70 genes were grouped into the “core”. The genes were

PIK3C=phosphatidylinositol-4,5-bisphosphate 3-kinase [EC:2.7.1.153];

PTEN=phosphatidylinositol-3,4,5-trisphosphate 3-phosphatase and dual-specificity protein phosphatase; KLK3=semenogelase [EC:3.4.21.77]; CTNNB1=catenin beta 1; BAD=Bcl-2-antagonist of cell death; BCL2=apoptosis regulator Bcl-2; CDK2=cyclin-dependent kinase 2 [EC:2.7.11.22]; NFKB1=nuclear factor NF-kappa-B p105 subunit; TCF7=transcription factor 7; PIK3R=phosphoinositide-3-kinase, regulatory subunit; HRAS=GTPase Hras; GSK3B=glycogen synthase kinase 3 beta [EC:2.7.11.26]; SOS=son of sevenless; htpG, HSP90A=molecular chaperone HtpG; EGF=epidermal growth factor; PDGFA=platelet-derived growth factor subunit A; EGFR, ERBB1=epidermal growth factor receptor [EC:2.7.10.1]; FGFR1=fibroblast growth factor receptor 1 [EC:2.7.10.1]; PDGFRA=platelet-derived growth factor receptor alpha [EC:2.7.10.1]; GRB2=growth factor receptor-binding protein 2; BRAF=B-Raf proto-oncogene serine/threonine-protein kinase [EC:2.7.11.1]; RAFT=RAF proto-oncogene serine/threonine-protein kinase [EC:2.7.11.1]; MAP2K1, MEK1=mitogen-activated protein kinase kinase 1 [EC:2.7.12.2]; MAP2K2, MEK2=mitogen-activated protein kinase kinase 2 [EC:2.7.12.2]; MAPK1_3=mitogen-activated protein kinase 1/3 [EC:2.7.11.24]; ATF4, CREB2=cyclic AMP-dependent transcription factor ATF-4; CASP9=caspase 9 [EC:3.4.22.62]; TP53, P53=tumor protein p53; AKT=RAC serine/threonine-protein kinase [EC:2.7.11.1]; IKBKA, IKKA, CHUK=inhibitor of nuclear factor kappa-B kinase subunit alpha [EC:2.7.11.10]; TCF7L1=transcription factor 7-like 1; TCF7L2=transcription factor 7-like 2; LEFT=lymphoid enhancer-binding factor 1; EP300, CREBBP, KAT3=E1A/CREB-binding protein [EC:2.3.1.48]; CCND1=cyclin Dl; INS=insulin; NFKBIA=NF-kappa-B inhibitor alpha; RELA=transcription factor p65; ERBB2, HER2; =receptor tyrosine-protein kinase erbB-2 [EC:2.7.10.1]; INSRR=insulin receptor-related receptor [EC:2.7.10.1]; IGF1R=insulin-like growth factor 1 receptor [EC:2.7.10.1]; PDGFRB=platelet-derived growth factor receptor beta [EC:2.7.10.1]; FGFR2=fibroblast growth factor receptor 2 [EC:2.7.10.1]; PDGFC_D=platelet derived growth factor C/D; IGF1=insulin-like growth factor 1; CREB1=cyclic AMP-responsive element-binding protein 1; PDPK1=3-phosphoinositide dependent protein kinase-1 [EC:2.7.11.1]; RB1=retinoblastoma-associated protein; E2F3=transcription factor E2F3; CDKN1B, P27, KIP1=cyclin-dependent kinase inhibitor 1B; CDKN1A, P21, CIP1=cyclin-dependent kinase inhibitor 1A; CCNE=cyclin E; MDM2=E3 ubiquitin-protein ligase Mdm2 [EC:2.3.2.27]; FOXO1=forkhead box protein O1; MTOR, FRAP, TOR=serine/threonine-protein kinase mTOR [EC:2.7.11.1]; IKBKB, IKKB=inhibitor of nuclear factor kappa-B kinase subunit beta [EC:2.7.11.10]; IKBKG, IKKG, NEMO=inhibitor of nuclear factor kappa-B kinase subunit gamma; KRAS, KRAS2=GTPase Kras; NRAS=GTPase Nras; NR3C4, AR=androgen receptor; TGFA=transforming growth factor, alpha; ARAF, ARAF1=A-Raf proto-oncogene serine/threonine-protein kinase [EC:2.7.11.1]; CREB5, CREBPA=cyclic AMP-responsive element-binding protein 5; CREB3; =cyclic AMP-responsive element-binding protein 3; NKX3-1=homeobox protein Nkx-3.1; E2F2=transcription factor E2F2; HSP90B, TRA1=heat shock protein 90 kDa beta; SRD5A2=3-oxo-5-alpha-steroid 4-dehydrogenase 2 [EC:1.3.1.22]; PDGFB=platelet-derived growth factor subunit B; and E2F1=transcription factor E2F1.

In a subsequent step, the core prostate cancer network was created in that the list of core prostate cancer genes was copy-pasted into the STRING database search page (http://string-db.org/cgi/input.pl?input_page_active_form=multiple_identifiers) to create a network:

http://bit.ly/28XP7HT (71 genes, option ‘minimum required interaction score’: low confidence (0.150), option ‘disable structure previews inside network bubbles’ switched on)

Thereafter the first layer of the prostate cancer network was created.

To create the first layer, ‘data settings’ and enter, in the field ‘2nd shell’: ‘no more than 20 interactors’ were chosen. The genes that have been added, became part of the first layer (91 genes−71 genes=20 genes).

In the next step, the second and outer layers of the prostate cancer network were created.

To create a second layer, these genes were entered into the STRING database search page, and chosen again for the option ‘2nd shell’: ‘no more than 50 interactors’. All new genes that pop up, became part of the second layer (50 genes).

In this example, the third layer (or, in this case, outer layer) consists of all genes in the human genome that are not part of either the core or the first layer. In the subsequent step, the genomic data were anonymized.

For anonymization a dataset with genomic data (e.g. expression data) for the complete genome (20,457 genes, according to the STRING database) of 100 individuals was used.

The core of 71 genes was not anonymized, because all the information from these prostate cancer related genes is required.

The first layer of 20 genes was anonymized by statistical anonymization, because the information from these genes might be important. More precisely this was done by generalizing or suppressing the values of these genes in order to achieve the k-anonymity and l-diversity properties, for chosen k (e.g. k=2) and l (e.g. l=3).

The second layer of 50 genes was be anonymized using homomorphic encryption, because the information from these genes might still be important. This method could be more convenient to apply when the layer has a bigger number of genes (e.g. greater or equal than 50).

The outer layer of 20,316 genes was anonymized by non-malleable encryption, because the information from these genes is not important for our specific study on prostate cancer.

1. A method for anonymization of genetic data from at least one individual, said method comprising the steps of:
providing genetic data from at least one individual;
choosing a disease to be studied;
determining at least one subset of the genetic data, said subset of genetic data being directly related to the disease to be studied;
assorting the remaining genetic data which are not directly related to the disease to be studied into multiple subsets grouped into more than one layer based on the proximity of these subsets to the genetic data which are directly related to the disease to be studied, wherein the proximity is preferably established based on a genome pathway network that corresponds to the genetic data;
anonymizing the more than one layer containing the subsets of genetic data not directly related to the disease to be studied.

2. The method according to claim 1, wherein the method further comprises analyzing the genetic data with respect to the disease to be studied.

3. The method according to claim 1, wherein the genetic data are selected from the group consisting of nucleotide sequences, Amplified Fragment Length Polymorphisms, Randomly Amplified Polymorphic DNA, Restriction Fragment Length Polymorphisms, Single Nucleotide Polymorphisms, Short Tandem Repeats and Variable Number Tandem Repeats, RNA, amino acid sequences, polypeptides, proteins and copy number data.

4. The method according to claim 1, wherein the number of layers is 2, 3, 4, 5, 6, 7, 8, 9, or 10.

5. The method according to claim 1, wherein the anonymizing is performed by using at least one technique selected from the group consisting of statistical anonymization, encryption and secure multiparty anonymization and computation.

6. The method according to claim 5, wherein statistical anonymization is selected from the group consisting of k-anonymity, l-diversity, t-closeness and δ-presence.

7. The method according to claim 5, wherein the encryption is selected from the group consisting of homomorphic encryption, searchable encryption and non-malleable encryption.

8. The method according to claim 1, wherein the different layers are anonymized by different techniques, preferably depending on the distance of the layers' subsets of genetic data to the subset of genetic data being directly related to the disease to be studied.

9. The method according to claim 1, wherein the subset of genetic data being directly related to the disease to be studied is selected from at least one database defining genes encoding polypeptides that were identified to be directly related to the disease to be studied.

10. The method according to claim 1, wherein genetic data of the first layer's subsets of genetic data are selected from the group of genes encoding polypeptides which are not directly related to the disease to be studied, but known to directly interact with one of the genes and/or polypeptides encoded by one of the genes of the genetic data which are directly related to the disease to be studied.

11. The method according to claim 10, wherein at least one of the first layer's subsets of genetic data is included into the subset of genetic data being determined to be directly related to the disease to be studied.

12. The method according to claim 11, wherein a subset of genetic data being in straight line to a given subset of genetic data is assorted into the layer next closer to the genetic data being directly related to the disease to be studied.

13. A computer program product for anonymizing genetic data, the computer program product comprising instructions which when carried out on a computer cause the computer to perform at least one step of a method for anonymizing genetic data of at least one individual, the method comprising the steps of:
providing genetic data from at least one individual;
choosing a disease to be studied;
determining at least one subset of the genetic data, said subset of genetic data being directly related to the disease to be studied;
assorting the remaining genetic data which are not directly related to the disease to be studied into multiple subsets grouped into more than one layer based on the proximity of these subsets to the genetic data which are directly related to the disease to be studied, wherein the proximity is preferably established based on a genome pathway network that corresponds to the genetic data;
anonymizing the more than one layer containing the subsets of genetic data not directly related to the disease to be studied.

14. A system for anonymizing genetic data, said system comprising:
a data interface configured to receive genetic data of at least one individual;
a user input interface configured to receive user input commands form a user input device for choosing a disease to be studied;
a processor configured for
determining subset(s) of genetic data from the genetic data of the at least one individual being directly related to the disease to be studied;
assorting the subsets of the genetic data that are not directly related to the disease to be studied into different layers based on the subsets' distance to the genetic data being directly related to the disease to be studied, wherein the distance is preferably established based on a genome pathway network that corresponds to the genetic data; and
anonymizing the layers that are not directly related to the disease to be studied or the genetic data present in the layers that are not directly related to the disease to be studied.

15. Use of the method according to claim 1, the computer program product according to claim 13 and/or the system according to claim 14 in one selected from the group consisting of genomics, genetics, bioinformatics research, transcriptomics, proteomics and systems biology or diagnosis.
